(19) United States
(12) Patent Application Publication
(10) Pub. No.: US 2008/0260671 A1
(43) Pub. Date: Oct. 23, 2008

(54) Depilatory Compositions

(75) Inventors: Frederic De La Torre, Hull (GB); Paul Andrew David Wilkinson, Hull (GB)

Correspondence Address: MERCHANT & GOULD PC, P.O. BOX 2903, MINNEAPOLIS, MN, 55402-0903, US

(73) Assignee: Reckitt Benckiser (UK) Limited, Slough (GB)

(21) Appl. No.: 11/629,221

(22) PCT Filed: Jun. 15, 2005
(86) PCT No.: PCT/GB05/02362

§ 371 Date: Aug. 13, 2007

(30) Foreign Application Priority Data
Jun. 15, 2004 (GB) 0413298.1

(51) Int. Cl. A61K 8/46; A61Q 9/04

(52) U.S. Cl. 424/70.1

(57) ABSTRACT

A depilatory composition comprising a depilatory compound having a thiol group and a quinone and/or phenol compound.

The present invention relates to depilatory compositions comprising a depilatory compound having a thiol group and to a method of depilation using such compositions.

Compositions for removing superfluous body hair are well known and are of various types. One type of composition requires initial heating before being applied to the skin in a generally molten state. It is then allowed to solidify before being removed from the skin together with unwanted hair.

Another type of depilatory composition is in the form of a composition, such as a cream, which can be applied to the skin, generally at room temperature. The cream includes a substance that degrades hair keratin. After the composition has been applied it is allowed to remain on the skin to degrade the hairs and then removed together with the degraded hairs.

Depilatory compositions of the type which degrades the hairs comprise a depilatory compound which is able to degrade the hairs. Depilatory compounds in common use, such as potassium thioglycolate, and other such compounds having a thiol group, have a disadvantage in that they have an unattractive smell. The composition may intrinsically have an unattractive smell which is noticed by a user when it is removed from a container. During use, however, the unattractive smell can increase due to reaction of the depilatory compound with the hair which is being or which has been degraded. This is particularly unpleasant for the consumer. It would be desirable to have depilatory compositions with a reduced smell, in particular a reduced smell when in use.

Although agents are known to reduce the malodour when added to depilatory compositions, such agents also reduce the efficacy of the depilatory compound. An example of such an agent is zinc oxide.

Furthermore depilatory compositions typically contain compounds which can irritate and even damage the skin. For example they typically contain sodium hydroxide to provide a high pH. The depilatory compositions are applied to the skin and allowed to act on the skin for a sufficient time to degrade the hairs. However, the compositions should not be allowed to act on the skin for longer than a certain time so as to reduce the irritant effect and possible damage to the skin. Although instructions are typically provided with depilatory compositions informing the user of the correct residence time, users do not always read them or follow the instructions correctly. It would therefore be desirable to have a composition which has an appropriate end-of-life indication after a suitable residence time so that a user knows when it is appropriate to remove the composition or which indicates when the composition is likely to have remained on the user's skin too long.

The present invention provides a depilatory composition comprising a depilatory compound having a thiol group, and a quinone and/or phenol compound.

We have surprisingly discovered that adding a quinone and/or a phenol compound to a depilatory composition comprising a depilatory compound having a thiol group may counteract the malodour of the depilatory composition and/or provide an end-of-use indication or indication that the composition should be removed from the skin by providing a colour change to the composition.

The quinone and/or phenol compound have surprisingly been found to reduce or remove the bad smell of the depilatory composition without substantially adversely affecting the ability of the depilatory compound to degrade hair. It has also been found that the quinone and/or phenol compound may cause the depilatory composition to exhibit a colour change after an appropriate time. For example, depilatory compositions typically are white. However, when a quinone and/or phenol compound is present in the composition it has surprisingly been found that the composition exhibits a colour change, for example turning to yellow and then to brown after about 10 minutes, which is an appropriate residence time on the skin. It is believed, although we are not bound by this theory, that this is caused by an oxidation reaction after the composition is exposed to air. The colour change can be controlled, for example, by altering the concentration of the quinone or phenol compound or by changing the nature of the compound. Furthermore including a reducing agent in the composition can assist in retarding the development of the colour change, thus enabling the timing of the change to be controlled. Inclusion of compounds, such as an amine or a metal-containing compound, which can react with the quinone or phenol compound, may give more intensity to the colour. Suitable metal containing compounds are lithium, potassium, sodium or magnesium in the form of hydroxides or silicates or in hectorite or montmorillonite structures.

The depilatory compound having a thiol group may be any compound capable of degrading keratin. Examples of such compounds are potassium thioglycolate, dithioerythritol, thioglycerol, thioglycol, thioxanthine, thiosalicylic acid, N-acetyl-L-cysteine, lipoic acid, sodium dihydrolipoate 6,8-dithioocatanoate, sodium 6,8-diothioocatanoate, a hydrogen sulphide salt, thioglycolic acid, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammoniumdithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazine, keratinase, guanidine thioglycolate, calcium thioglycolate and/or cysteamine. A single compound or a mixture of two or more compounds may be used.

Preferably the depilatory compound is potassium thioglycolate.

The depilatory compound is preferably present in the composition in an amount of from 1 to 10 wt % based on the total weight of the composition, more preferably from 2 to 7 wt %.

The quinone and/or phenol compound is, for example, a quinone or phenol antioxidant.

Preferred quinone and/or phenol compounds are of the formula:

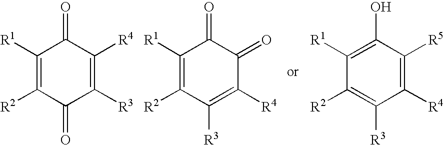

wherein R¹R², R³, R⁴ and R⁵, which may be identical or different, are each hydrogen, halo, hydroxyl, amino, nitro, alkyl or a group containing an ester linkage, it being possible for the quinone or phenol ring to be fused to an aliphatic or aromatic ring which may itself optionally have one or more substituents selected from halo, hydroxyl, amino, nitro, alkyl or a group containing an ester linkage.

Examples of the halo group are chlorine, fluorine and/or bromine.

The amino group may be a primary amine group or a secondary or tertiary amine group in which the hydrogen atoms of the primary amine group have been substituted by, for example, one or more alkyl groups, in particular alkyl groups containing from 1 to 6 carbon atoms.

The alkyl group is desirably a group containing up to 10 carbon atoms, preferably from 1 to 6 carbon atoms and more preferably from 1 to 4 carbon atoms. Examples of suitable alkyl groups are methyl, ethyl, n-propyl, i-propyl, n-butyl, sec-butyl, t-butyl, pentyl and hexyl. The alkyl group may be straight, branched or cyclic.

The group containing an ester linkage is desirably of formula —COO—R⁶ wherein R⁶ is an alkyl or alkenyl group, for example containing from 1 to 18 carbon atoms, in particular from 1 to 4 carbon atoms. Examples of suitable alkyl and alkenyl groups are methyl, ethyl, n-propyl, i-propyl, n-butyl, sec-butyl, t-butyl and allyl.

Desirably the quinone compound, particularly of the first two formulae depicted above, contains one, two, three or four substituents. Desirably the phenol compound, and especially the third compound depicted above, contains one, two, three, four or five substituents. Desirably the phenol compound contains a total of one, two or three hydroxyl groups on the aromatic ring.

Examples of suitable quinone compounds are chloranil, chloranilic acid, 1,4-naphthoquinone, 2,6-dichloro-p-benzoquinone, 2-chloroanthroquinone, 2,3-dichloro-1,4-naphthoquinone, o-benzoquinone, 2,6-dimethyl-p-benzoquinone, 2,3,5,6-tetramethyl-p-benzoquinone, 1,4-naphthoquinone, 1-nitroanthroquinone, 1-aminoanthroquinone, 2-amoneoanthroquinone, 4,5-dichloro-1-nitroanthroquinone, 1,2-dihydroxyanthroquinone, phenanthrenequinone, t-butyl hydroquinone, hydroquinone, 2,5-di-tert-butyl hydroquinone, propyl gallate and/or butylated hydroxyanisole.

Preferred quinone compounds are propyl gallate, 2,6-di-tert-butyl-p-cresol, butylated hydroxyanisole, and/or t-butyl hydroquinone.

Most desirably the quinone compound is t-butyl hydroquinone (mono t-butyl hydroquinone).

Examples of the phenol compound are 2,6-di-tert-butyl-p-cresol, resorcinol, hexyl resorcinol, catechol, pyrogallol, phloroglucinol,4-chlororesorcinol, resveratrol, nordihydroguaiaretic acid and/or quercetin.

The quinone compound and/or phenol compound may be present in an isomeric form. In particular, the quinone compound and/or phenol compound can partially or fully be in a tautomer form such as a keto-enol form, particularly under the alkaline conditions of a depilatory composition.

The quinone and/or phenol compound may be used singly or a mixture of two or more compounds such as two or more quinone compounds, two or more phenol compounds or a mixture of at least one quinone compound and at least one phenol compound.

The composition preferably comprises from 0.001 to 10 wt % of the quinone and/or phenol compound based on the total weight of the composition, preferably from 0.01 to 1 wt %, most preferably from 0.05 to 0.1 wt %.

The depilatory composition may, if desired, comprise further components.

The depilatory composition may, for example, comprise components which accelerate the keratin degradation reaction such as urea thiourea, dithioerythritol, dimethyl isosorbide (DMI), ethoxydiglycol (Transcutol) or methyl propyl diol (MT diol). The composition desirably comprises up to 15 wt % of the accelerator based on the total weight of the composition, preferably from 5 to 15 wt % and more preferably from 7 to 10 wt %.

The composition may comprise a source of alkalinity, for example an alkali metal hydroxide such as sodium hydroxide or potassium hydroxide. Desirably the pH of the composition of the present invention is at least 12, more preferably at least 12.4.

Preferably the alkali metal hydroxide is present in an amount of at least 0.001 mol/100 g of composition, preferably in an amount of at least 0.01 mol/100 g composition.

The depilatory composition of the present invention may comprise water, suitably in an amount of at least 40 or 50 wt % based on the total weight of the composition, more preferably at least 60 wt % and even more preferably at least 68 wt %. Preferably the water is present in an amount of up to 95 wt % based on total weight of composition, more preferably up to 85 wt % and even more preferably up to 75 wt %.

The depilatory composition may, for example, be in the form of an oil-in-water emulsion, a water-in-oil emulsion, a microemulsion, a multiple emulsion, a lotion, cream, gel or foam.

The depilatory composition may comprise further components such as perfumes, oils, pigments, clays, fillers such as lithium sodium magnesium silicate, magnesium trisilicate and titanium dioxide.

If the depilatory composition is in the form of a cream it may, for example, use a conventional cream base, such as a mixture of polypropylene glycol ester and cetostearyl alcohol.

The composition of the present invention may be prepared, for example, by mixing the various components together, preferably at a temperature not exceeding 85° C., and preferably at a temperature of from 65° C. to 85° C. More preferably all of the components excluding the depilatory compound are first mixed together at this elevated temperature, the resultant composition is actively or passively cooled and the depilatory compound added to the cooled composition at a temperature of from 15° C. to 40° C., preferably at ambient temperature (e.g. about 20° C.). The source of alkalinity such as the alkali metal hydroxide may be added at any stage of the process, but preferably after the depilatory compound is added. The quinone and/or the phenol compound can also be added at any stage of the process. It is generally added in the form of a solution in a solvent such as glycol, and is preferably added at the same time or with the depilatory compound.

The present invention also provides a method of depilation comprising:
a. applying to the skin a composition as defined in above;
b. allowing the composition a residence time on the skin to degrade hairs;
c. at the end of the residence time removing the composition and depilated hairs from the skin; and
d. rinsing the skin.

Preferably the residence time on the skin is from 3 to 10 minutes. Desirably the appropriate residence time is coordinated with a suitable colour change of the composition such that a user of the composition would know when it is appropriate to remove the composition. The composition may be removed from the skin using a spatula or scraper device. Where the colour change is gradual, the user is preferably provided also with an indicator chart or card showing the colour which the composition should attain before the composition and depilated hairs are removed from the skin.

Hence the present invention provides a method of depilation comprising;
a. providing a composition according to the invention as defined above in a container,
b. applying the composition to the skin such that the composition is exposed to the air,
c. allowing the composition to reside on the skin exposed to air until a colour change of the composition occurs,
d. removing the composition and depilated hairs from the skin until a colour change of the composition has occurred, and
e. rinsing the skin.

Suitably the composition will be provided in a sealed single use ampule or in tube or a bottle with a replaceable cap such that the composition is not exposed to air on storage.

The purpose of the container is to prevent excessive contact between the composition and air prior to exposure on the skin of the consumer.

The present invention further provides the use of a quinone and/or phenol compound in a depilatory composition comprising a depilatory compound having a thiol group to reduce the malodour of said depilatory compound when said composition is in use.

The present invention additionally provides the use of a quinone and/or phenol compound in a depilatory composition comprising a depilatory compound having a thiol group to provide a colour change to said composition after said composition has been applied to the skin of a user for a predetermined time.

The use of the accelerant such as a monovalent or divalent hydroxide (for example Ca(OH)₂ or KOH) advantageously results in accelerated hair removal action of the depilatory composition. However, the use of monovalent or divalent hydroxides (in particular Ca(OH)₂) in the composition results in a more sudden burst of odour which is disadvantageous to the end user of the depilatory composition. Furthermore, the use of such accelerants results in the composition requiring a shorter residence time on the users skin. A problem associated with such compositions requiring a shorter residence time is that the user often leaves the composition on the skin for longer than required, thereby causing irritation.

Accordingly, there is further provided a use of a quinone and/or phenol compound in combination with a monovalent and/or a divalent hydroxide in a depilatory composition comprising a depilatory compound having a thiol group, to provide a colour change to the composition after the composition has been exposed to the air whilst on the skin of a user for a predetermined time.

A suitable hydroxide may be Ca(OH)₂; a typical colour change on the skin of a user when such a hydroxide is used may be from a substantially white colour when the composition is applied to a users skin, through yellow to brown after about 10 minutes; this being the appropriate residence time on the skin.

The hydroxide may also be KOH. A typical colour change when KOH is used would be from substantially white to substantially pink within about a 10 minute timeframe. It is, of course, envisaged that other hydroxides would produce different colour changes, each colour change used being dependent on the hydroxide included.

The quinone, phenol compound and depilatory compound are substantially as described hereinbefore.

Preferably, the depilatory composition includes the hydroxide in an amount in the range 1.5% to 8% by weight of the composition. It is particularly preferred that the hydroxide is in the range 2.5% to 6.5% by weight of the composition, further preferably 3.0 to 4.5% by weight.

The present invention is further illustrated in the following Examples:

### EXAMPLE 1

A depilatory composition comprising 0.1 wt % tert-butyl hydroquinone (TBHQ) was prepared. The composition was as follows:

| | | 500 g Lab | |
|---|---|---|---|
| Ingredient (Trade Name) | % w/w | Sample (g) | |
| Ceteareth-20 | 2.2 | 11 | |
| Cetearyl alcohol | 5.5 | 27.5 | |
| PPG-15 Stearyl Ether | 1.5 | 7.5 | |
| Deionised Water | 47.4 | 237 | |
| Calcium Hydroxide | 2.9 | 14.5 | |
| Sodium Gluconate | 0.1 | 0.5 | |
| Magnesium Trisilicate | 0.5 | 2.5 | |
| Titanium dioxide/propylene glycol | 0.6 | 3 | |
| paste | | | |
| Deionised Water | 20.5 | 102.5 | |
| Urea | 8 | 40 | |
| Lithium Magnesium Sodium Silicate | 0.5 | 2.5 | |
| Acrylates Copolymer | 0.1 | 0.5 | |
| Potassium thioglycolate | 10 | 50 | |
| Crosilk Liquid (Silk Extract) | 0.1 | 0.5 | |
| TBHQ | 0.1 | 0.5 | |
| TOTALS | 100% | 500 | g |

The composition was exposed to air following removal from an air-tight container. The initial colour was white. After 2 minutes the composition changed colour to Pantone no. 468 C/55C Matt, after 3 minutes to 7501C/106C Matt, after 5 minutes to 467C/55C Matt and after 10 minutes to beige (no good Pantone match).

A comparative example was prepared without TBHQ (replaced by deionised water as in comparative Example 1—see below) This also had an initial colour which was white. The comparative example exhibited no colour change on exposure to air, following the experimental procedure as for Example 1.

### EXAMPLE 2

A depilatory composition comprising 0.05 wt % TBHQ was prepared similarly as in Example 1. The composition was as follows:

| | | 500 g Lab | |
|---|---|---|---|
| Ingredient (Trade Name) | % w/w | Sample (g) | |
| Ceteareth-20 | 2.2 | 11 | |
| Cetearyl alcohol | 5.5 | 27.5 | |
| PPG-15 Stearyl Ether | 1.5 | 7.5 | |
| Deionised Water | 47.45 | 237.25 | |
| Calcium Hydroxide | 2.9 | 14.5 | |
| Sodium Gluconate | 0.1 | 0.5 | |
| Magnesium Trisilicate | 0.5 | 2.5 | |
| Titanium dioxide/propylene glycol | 0.6 | 3 | |
| paste | | | |
| Deionised Water | 20.5 | 102.5 | |
| Urea | 8 | 40 | |
| Lithium Magnesium Sodium Silicate | 0.5 | 2.5 | |
| Acrylates Copolymer | 0.1 | 0.5 | |
| Potassium thioglycolate | 10 | 50 | |
| Crosilk Liquid (Silk Extract) | 0.1 | 0.5 | |
| TBHQ | 0.05 | 0.25 | |
| TOTALS | 100% | 500 | g |

### COMPARATIVE EXAMPLE 1

A depilatory composition not containing any quinone or phenol compound was prepared similarly as in Example 1. The composition was as follows:

| | | 500 g Lab | |
|---|---|---|---|
| Ingredient (Trade Name) | % w/w | Sample (g) | |
| Ceteareth-20 | 2.2 | 11 | |
| Cetearyl alcohol | 5.5 | 27.5 | |
| PPG-15 Stearyl Ether | 1.5 | 7.5 | |
| Deionised Water | 47.5 | 237.5 | |
| Calcium Hydroxide | 2.9 | 14.5 | |
| Sodium Gluconate | 0.1 | 0.5 | |
| Magnesium Trisilicate | 0.5 | 2.5 | |
| Titanium dioxide/propylene glycol | 0.6 | 3 | |
| paste | | | |
| Deionised Water | 20.5 | 102.5 | |
| Urea | 8 | 40 | |
| Lithium Magnesium Sodium Silicate | 0.5 | 2.5 | |
| Acrylates Copolymer | 0.1 | 0.5 | |
| Potassium thioglycolate | 10 | 50 | |
| Crosilk Liquid (Silk Extract) | 0.1 | 0.5 | |
| TOTALS | 100% | 500 | g |

The compositions of the Examples were compared to demonstrate the ability of the compositions of the present invention to reduce malodour when in contact with human hair as compared with the composition of the Comparative Example. Each sample was mixed with finely cut human hair of Asian origin in separate petri dishes at a ratio of 1:10 (Hair to Cream) and placed inside separate 1200 cm³ bell jars. These were then sealed using vacuum grease, and a stopper, and labelled accordingly with coded numbers. The 1:10 ratio equated to each bell jar containing 0.5 g of hair to 5 g of test cream.

The bell jars were then left for a period of 15 minutes to allow for any malodour generation to collect in the head-space of the bell jar.

Volunteers were asked to remove the stopper, and first smell the reference sample which was identical to the composition of comparative Example 1—which they were asked to assign the number 10 for level of malodour.

The volunteers were then asked to smell the three test samples and mark each sample on the scale provided for presence of malodour

The test sheets randomised the order in which the samples were tested. This randomisation was done using a Latin square technique.

Two repetitions were carried out. Each repetition involved 30 volunteers taking a sniff-test of the reference sample and then the three test samples, filling out the score sheet accordingly as they progressed. For each repetition fresh samples were made.

The scale the volunteers were presented with was from 0 to 10 with the two following references:
0—No Malodour
10—Malodour at the same level as the reference sample.

### Repetition One:

TABLE 1

| | Volunteer | Mean Malodour Score | Result |
|---|---|---|---|
| Sample | Size | given by volunteer | Group |
| Comparative | 30 | 8.20 | A |
| Example 1 | | | |
| Example 1 | 30 | 2.80 | B |
| Example 2 | 30 | 2.73 | B |

L.S.D: 1.05

### Repetition Two:

TABLE 2

| | Volunteer | Mean Malodour Score | Result |
|---|---|---|---|
| Sample | Size | given by volunteer | Group |
| Comparative | 30 | 7.00 | A |
| Example 1 | | | |
| Example 1 | 30 | 2.87 | B |
| Example 2 | 30 | 2.77 | B |

L.S.D: 1.05

The results presented were analysed using analysis of variance and L.S.D. at 5% to identify significant differences.

The difference between the malodour levels with the same letter is not significant. However the two result groups assigned different letters have a significant difference between them.

Both repetition results show that there is a statistically significant reduction in malodour generation between samples containing TBHQ at both the percentages of 0.1% and 0.05% compared with the standard sample containing no TBHQ.

### EXAMPLE 3

A depilatory composition comprising 0.09 wt % TBHQ was prepared similarly as in Example 1. The composition was as follows:

| Ingredient | |
|---|---|
| (Chemical name) | % w/w |
| Cetearyl Alcohol | 1.5 |
| Ceteareth-20 | 0.2 |
| Petrolatum Codex | 0.5 |
| Paraffin wax | 0.5 |
| Shea Butter | 0.5 |
| Deionised Water | 20 |
| Deionised Water | 30.5 |
| NaOH 47% | 5.6 |
| Carbomer | 0.5 |
| Urea | 8 |
| Na Silicate | 3 |
| Potassium Thioglycolate 30% | 15 |
| Deionised Water | 13.81 |
| TBHQ | 0.09 |
| Propylene Glycol | 0.3 |
| TOTAL | 100% |

The depilatory composition resulted in a reduction of bad odour when compared to depilatory compositions which do not contain TBHQ.

### EXAMPLE 4

A Standard Base Formulation depilatory composition not containing any quinone or phenol compound was prepared similarly as in Example 1. The composition was as follows:

| | | 100 g Lab | |
|---|---|---|---|
| Ingredient (Trade Name) | % w/w | Sample (g) | |
| Deionised Water | 45.895 | 45.895 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolymer | 0.1 | 0.1 | |
| Potassium thioglycolate | 12.9 | 12.9 | |
| Potassium Hydroxide (50% | 1 | 1 | |
| Solution) | | | |
| TOTALS | 100% | 100 | g |

Five further comparative depilatory compositions were prepared similarly as in Example 1. Each composition contained 1% 2 Keto-L-Gulonic Acid (In Water), 1% Pyrogallol (In Propylene Glycol), 1% Hydroquinone (In Propylene Glycol), 0.05% Hydroquinone (In Propylene Glycol), 0.05% TBHQ(In Propylene Glycol). The compositions were as follows:

1% 2 Keto-L-Gulonic Acid (In Water)

| | | 100 g Lab | |
|---|---|---|---|
| Ingredient (Trade Name) | % w/w | Sample (g) | |
| Deionised Water | 38.895 | 38.895 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolomer | 0.1 | 0.1 | |
| Potassium thioglycolate | 12.9 | 12.9 | |
| Potassium Hydroxide (50% | 1 | 1 | |
| Solution) | | | |
| 2 Keto-L-Gulonic Acid | 1 | 1 | |
| Deionised Water | 6 | 6 | |
| TOTALS | 100% | 100 | g |

1% Pyrogallol (In Propylene Glycol)

| | | 100 g Lab | |
|---|---|---|---|
| Ingredient (Trade Name) | % w/w | Sample (g) | |
| Deionised Water | 38.895 | 38.895 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolymer | 0.1 | 0.1 | |
| Potassium thioglycolate | 12.9 | 12.9 | |
| Potassium Hydroxide (50% | 1 | 1 | |
| Solution) | | | |
| Pyrogallol | 1 | 1 | |
| Propylene Glycol | 6 | 6 | |
| TOTALS | 100% | 100 | g |

1% Hydroquinone (In Propylene Glycol)

| | | 100 g Lab | |
|---|---|---|---|
| Ingredient (Trade Name) | % w/w | Sample (g) | |
| Deionised Water | 38.895 | 38.895 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolymer | 0.1 | 0.1 | |
| Potassium thioglycolate | 12.9 | 12.9 | |
| Potassium Hydroxide (50% | 1 | 1 | |
| Solution) | | | |
| Hydroquinone | 1 | 1 | |
| Propylene Glycol | 6 | 6 | |
| TOTALS | 100% | 100 | g |

0.05% Hydroquinone (In Propylene Glycol)

| | | 100 g Lab | |
|---|---|---|---|
| Ingredient (Trade Name) | % w/w | Sample (g) | |
| Deionised Water | 39.845 | 39.845 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolymer | 0.1 | 0.1 | |
| Potassium thioglycolate | 12.9 | 12.9 | |
| Potassium Hydroxide (50% | 1 | 1 | |
| Solution) | | | |
| 0.05% Hydroquinone | 0.05 | 0.05 | |
| Propylene Glycol | 6 | 6 | |
| TOTALS | 100% | 100 | g |

0.05% TBHQ(In Propylene Glycol)

| | | 100 g Lab | |
|---|---|---|---|
| Ingredient (Trade Name) | % w/w | Sample (g) | |
| Deionised Water | 45.545 | 45.545 | |
| Calcium Hydroxide | 3.75 | 3.75 | |
| Sodium Gluconate | 0.1 | 0.1 | |
| Magnesium Trisilicate | 0.5 | 0.5 | |
| Ceteareth-20 | 1.43 | 1.43 | |
| Cetearyl alcohol | 3.6 | 3.6 | |
| PPG-15 Stearyl Ether | 1 | 1 | |
| Thick Mineral Oil | 0.1 | 0.1 | |
| Sweet Almond Oil | 0.1 | 0.1 | |
| Deionised Water | 21.325 | 21.325 | |
| Urea | 8 | 8 | |
| Lithium Magnesium Sodium Silicate | 0.2 | 0.2 | |
| Acrylates Copolymer | 0.1 | 0.1 | |
| Potassium thioglycolate | 12.9 | 12.9 | |
| Potassium Hydroxide (50% | 1 | 1 | |
| Solution) | | | |
| 0.05% TBHQ | 0.05 | 0.05 | |
| Propylene Glycol | 0.3 | 0.3 | |
| TOTALS | 100% | 100 | g |

The compositions of the examples were compared to demonstrate the ability of the compositions of the present invention to reduce malodour when in contact with human hair as compared with the composition of the standard base formulation. Malodour tests were carried out similarly to the tests in Example 2.

### Results:

| | Results | | |
|---|---|---|---|
| | Volunteer | Mean | Result |
| Ingredient | Size | Score | Group |
| Blind Standard | 27 | 7.89 | A |
| (Standard | | | |
| Formulation) | | | |
| 2-Keto-L-Gulonic | 27 | 7.63 | AB |
| Acid (Present at 1% w/w) | | | |
| Hydroquinone(Present | 27 | 6.52 | BC |
| at 1% w/w) | | | |
| Hydroquinone(Present | 27 | 6.00 | C |
| at 0.05% w/w) | | | |
| Pyrogallol(Present | 27 | 5.63 | C |
| at 1% w/w) | | | |
| TBHQ (Present at | 27 | 3.93 | D |
| 0.05% w/w) | | | |

The results indicate that there is a statistically significant reduction in malodour generation between depilatory compounds that contain quinone or a phenol compound than depilatory compounds that do not.

1. A depilatory composition comprising an effective amount of a depilatory compound having a thiol group, and an effective amount of a malodour reducing compound selected from the group consisting of a quinone compound, a phenol compound or mixtures thereof.

2. The composition of claim 1 wherein the depilatory compound is potassium thioglycolate, dithioerythritol, thioglycerol, thioglycol, thioxanthine, thiosalicylic acid, N-acetyl-L-cysteine, reduced lipoic acid, sodium dihydrolipoate 6,8-dithiooctanoate, sodium 6,8-dithiooctanoate, a hydrogen sulphide salt, thioglycolic acid, 2-mercaptopropionic acid, 3-mercaptopropionic acid, thiomalic acid, ammonium thioglycolate, glyceryl monothioglycolate, monoethanolamine thioglycolate, monoethanolamine thioglycolic acid, diammoniumdithiodiglycolate, ammonium thiolactate, monoethanolamine thiolactate, thioglycolamide, homocysteine, cysteine, glutathione, dithiothreitol, dihydrolipoic acid, 1,3-dithiopropanol, thioglycolamide, glycerylmonothioglycolate, thioglycolhydrazine, keratinase, guanidine thioglycolate, calcium thioglycolate, cysteamine or mixtures thereof.

3. The composition according to claim 1 wherein the depilatory compound is potassium thioglycolate.

4. The composition of claim 1 wherein the phenol compound comprises the formula: 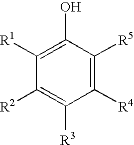
wherein R¹, R², R³, R⁴ and R⁵, which may be identical or different, are each hydrogen, halo, hydroxyl, amino, nitro, alkyl or a group containing an ester linkage, it being possible for the phenol ring to be fused to an aliphatic or aromatic ring which may itself optionally have one or more substituents selected from halo, hydroxyl, amino, nitro, alkyl or a group containing an ester linkage.

5. The composition of claim 1 in which the quinone compound is chloranil, chloranilic acid, 1,4-naphthoquinone, 2,6-dichloro-p-benzoquinone, 2-chloroanthroquinone, 2,3-dichloro-1,4-naphthoquinone, o-benzoquinone, 2,6-dimethyl-p-benzoquinone, 2,3,5,6-tetramethyl-p-benzoquinone, 1,4-naphthoquinone, 1-nitroanthroquinone, 1-aminoanthroquinone, 2-amoneoanthoroquinone, 4,5-dichloro-1-nitroantrhtoquinone, 1,2-dihydroxyanthroquinone, phenanthrenequinone, t-butyl hydroquinone, hydroquinone, 2,5-di-tert-butyl hydroquinone, propyl gallate, butylated hydroxyanisole or mixtures thereof.

6. The composition of claim 1 in which the quinone compound is propyl gallate, 2,6-di-tert-butyl-p-cresol, butylated hydroxyanisole, t-butyl hydroquinone or mixtures thereof.

7. The composition of claim 6 wherein the quinone compound is t-butyl hydroquinone.

8. The composition of claim 1 wherein the phenol compound is 2,6-di-tert-butyl-p-cresol, resorcinol, hexyl resorcinol, catechol, pyrogallol, phloroglucinol, 4-chlororesorcinol, resveratrol, nordihydroguaiaretic acid, quercetin or mixtures thereof.

9. The composition of claim 1 which comprises from 1 to 10 wt % of the depilatory compound based on the total weight of the composition.

10. The composition of claim 1 which comprises from 0.001 to 10 wt % of the malodour reducing compound based on the total weight of the composition.

11. The composition of claim 1 having a pH of at least 12.

12. A method of depilation comprising:
a. applying to the skin the composition of claim 1;
b. allowing the composition a residence time on the skin to degrade hairs;
c. at the end of the residence time removing the composition and depilated hairs from the skin; and
d. rinsing the skin.

13. A method of depilation comprising:
a. applying the composition of claim 1 to the skin such that the composition is exposed to the air,
b. allowing the composition to reside on the skin exposed to air until a colour change of the composition occurs,
c. removing the composition and depilated hairs from the skin after the colour change of the composition has occurred, and
d. rinsing the skin.

14. The composition of claim 1 wherein the quinone compound comprises the formula: 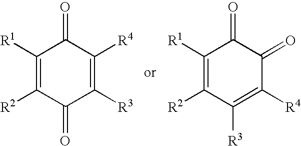
wherein R¹, R², R³, R⁴ and R⁵, which may be identical or different, are each hydrogen, halo, hydroxyl, amino, nitro, alkyl or a group containing an ester linkage, it being possible for the quinone ring to be fused to an aliphatic or aromatic ring which may itself optionally have one or more substituents selected from halo, hydroxyl, amino, nitro, alkyl or a group containing an ester linkage.

15-16. (canceled)
